(19) United States
(12) Patent Application Publication
(10) Pub. No.: US 2012/0064133 A1
(43) Pub. Date: Mar. 15, 2012

(54) Multiparticulate Controlled-Release Selective Serotonin Reuptake Inhibitor Formulations

(76) Inventors: Ishwar Chauhan, Voorhees, NJ (US); Siva Rama Krishna Nutalapati, Princeton, NY (US)

(21) Appl. No.: 13/321,571

(22) PCT Filed: May 24, 2010
(86) PCT No.: PCT/US10/35918

§ 371 Date: Nov. 21, 2011

Related U.S. Application Data
(60) Provisional application No. 61/181,767, filed on May 28, 2009.

(51) Int. Cl. A61K 31/15; A61P 9/14; A61P 25/00; A61K 47/14; A61K 47/42; A61K 47/38; A61K 47/46; A61K 47/32; A61K 9/00; A61P 25/24

(52) U.S. Cl. 424/400; 514/640; 514/773; 514/781; 514/783; 514/772.5; 514/772.6; 514/787; 514/786

(57) ABSTRACT

Multiparticulate controlled-release selective serotonin reuptake inhibitor (SSRI) formulations for oral administration, which comprise subunits of a SSRI or a pharmaceutically acceptable salt thereof which are not coated with a rate-controlling polymer, are provided. Methods for preparation and use of these formulations are also provided.

This patent application claims the benefit of priority from U.S. Provisional Application Ser. No. 61/181,767, filed May 28, 2009, teachings of which are herein incorporated by reference in their entirety.

### BACKGROUND OF THE INVENTION

Selective serotonin reuptake inhibitors (SSRIs) are a class of compounds administered for treatment of depression, anxiety disorders, and some personality disorders. They are also typically effective and used in treating premature ejaculation problems as well as some cases of insomnia. SSRIs increase the extracellular level of serotonin by inhibiting its reuptake into presynaptic cells, thereby increasing the level of serotonin which can bind to postsynaptic receptors. Examples of SSRIs include, but not limited to citalopram, clomipramine, fluoxetine, fluvoxamine, paroxetine, sertraline, trazodone, venlafaxine and zimeldine are used in the treatment of depression.

U.S. Pat. No. 7,465,462 discloses a multiparticulate controlled-release selective serotonin reuptake inhibitor (SSRI) formulation for oral administration. In this formulation, subunits of a SSRI or a pharmaceutically acceptable salt thereof are coated with rate-controlling polymer which allows controlled-release of the SSRI over a period of not less than about 12 hours following oral administration.

WO 05/048979 discloses a modified release pharmaceutical composition consisting of casing comprising at least two microtablets, which are coated with rate controlling agent(s) optionally in combination with auxiliary pharmaceutical excipient(s). Each micro tablet comprises core subunits comprising pharmaceutical active ingredient and rate controlling agent(s). Either the core subunits or the compressed micro tablets are also coated with rate controlling agents. Included as examples of active agents are SSRIs such as fluvoxamine maleate.

Published U.S. Application No. 2008/0138411 discloses a modified release pharmaceutical composition for oral administration, suitable for once daily dosing, comprising from about 4.0% (w/w) to about 8.0% (w/w) of a form of at least one selective serotonin re-uptake inhibitor, from about 10% (w/w) to about 40% (w/w) of at least one release rate controlling polymer, from about 40% (w/w) to about 60% (w/w) of at least one diluent, from about 0% (w/w) to about 5% (w/w) of at least one binder, from about 0% (w/w) to about 5.0% (w/w) of at least one lubricant, from about 0% (w/w) to about 6.0% (w/w) of at least one surfactant, from about 0% (w/w) to about 6.0% (w/w) of at least one solubilizing agent, from about 0% (w/w) to about 6.0% (w/w) of at least one bioavailability enhancer, and from about 0% (w/w) to about 6.0% (w/w) of at least one acidifying agent.

### SUMMARY OF THE INVENTION

An aspect of the present invention relates to a controlled-release selective serotonin (5HT) reuptake inhibitor (SSRI) oral formulation which is not coated with a rate-controlling polymer.

Another aspect of the present invention relates to a method for formulating a controlled-release SSRI formulation for oral administration without a rate-controlling polymer.

Another aspect of the present invention relates to a method for treating a subject in need of a SSRI comprising orally administering to the subject a controlled-release SSRI formulation which is not coated with a rate-controlling polymer.

### DETAILED DESCRIPTION OF THE INVENTION

The present invention provides a multiparticulate controlled-release selective serotonin reuptake inhibitor (SSRI) formulation for oral administration, which comprises subunits of a SSRI or a pharmaceutically acceptable salt thereof which are not coated with a rate-controlling polymer. The formulation of the present invention provides for controlled-release of the SSRI over a period of not less than about 12 hours following oral administration. Also provided in the present invention are methods for formulating these multiparticulate controlled-release selective serotonin reuptake inhibitor (SSRI) formulations and methods for their use in administering to subjects in need thereof.

The formulation of the present invention may comprise any active agent which selectively inhibits serotonin reuptake. Examples of SSRIs for use in the formulations of the present invention include, but are not limited to, citalopram, clomipramine, fluoxetine, fluvoxamine, paroxetine, sertraline, trazodone, venlafaxine and zimeldine, all of which inhibit serotonin reuptake to various degrees. By SSRI, as used herein, it is meant to be inclusive of all pharmaceutically acceptable salt forms, crystalline forms, amorphous form, polymorphic forms, solvates, and hydrates unless specifically indicated otherwise. Examples include, but are in no way limited to, selective serotonin reuptake inhibitor (SSRI) free base, selective serotonin reuptake inhibitor (SSRI) benzoate, selective serotonin reuptake inhibitor (SSRI) citrate, selective serotonin reuptake inhibitor (SSRI) glutarate, selective serotonin reuptake inhibitor (SSRI) hydrobromide, selective serotonin reuptake inhibitor (SSRI) hydrochloride, selective serotonin reuptake inhibitor (SSRI) hydrogen phosphate, selective serotonin reuptake inhibitor (SSRI) dihydrogen phosphate, selective serotonin reuptake inhibitor (SSRI) lactate, selective serotonin reuptake inhibitor (SSRI) mandelate, selective serotonin reuptake inhibitor (SSRI) maleate, selective serotonin reuptake inhibitor (SSRI) mesylate, selective serotonin reuptake inhibitor (SSRI) oxalate, selective serotonin reuptake inhibitor (SSRI) sulfate, and a hydrate, solvate, polymorph, or non-crystalline form of the foregoing. Thus, the SSRI can be present in the form of a free base or in the form of a pharmaceutically acceptable salt such as, but not limited to, the hydrochloride salt, the sulfate salt, the succinate salt or a maleate form. Further, the SSRI, where applicable, may be present either in the form of one substantially optically pure enantiomer or as a mixture, racemic or otherwise, of enantiomers.

By “subunit” or “subunits”, as used herein, it is meant to include granules, pellets, microtablets, minitablets and microcaplets. The mean diameter for granules, pellets or minitablets is generally greater than about 250 micrometers and the mean length of microtablets or microcaplets ranges from at least about 250 micrometers. The mean diameter for granules, pellets or minitablets generally ranges from at least about 250 micrometers up to about 7500 micrometers where as the mean length of microtablets or microcaplets ranges from at least about 250 micrometers up to about 1 centimeter. In one embodiment of the present invention, the mean diameter or mean length of the subunit is greater than about 500 micrometers. In another embodiment of the present invention, the mean diameter or mean length of the subunit is greater than about 1500 micrometers. In another embodiment of the present invention, the mean diameter or mean length of the subunits is from about 250 to about 7500 micrometers. In another embodiment of the present invention, the mean diameter or mean length of the subunits is from about 400 to about 5000 micrometers. In another embodiment of the present invention, the mean diameter or mean length of the subunits is from about 500 to about 4500 micrometers.

Subunits used in the formulations of the present invention comprise a core of a SSRI or a pharmaceutically acceptable salt thereof compressed with a release-retarding material. Subunits used in the formulations of the present invention are not coated with any rate-controlling polymer. Instead, the solid, oral controlled-release selective serotonin reuptake inhibitor (SSRI) dosage compositions generally comprise selective serotonin reuptake inhibitor (SSRI) and a release-retarding material(s) wherein the release-retarding material(s) is in the form of a matrix and not as a coating. The combination of selective serotonin reuptake inhibitor (SSRI) and release-retarding material(s) can be in the form of a monolithic tablet, a layered tablet; or subunit form such as a granule, a microtablet, a minitablet, a microcaplet, a pellet (as used herein “pellet” means a spherical granule prepared by extrusion and spheronization, and is equivalent to bead, spheroid, and microsphere).

The release-retarding material(s) of the matrix are selected so as to achieve, in combination with other stated properties herein, a desired in vitro or in vivo release profile.

Examples of release-retarding matrix material(s) to be used in a SSRI containing subunit matrix of the present invention, include, but are not limited to, an acrylic polymer, an alkylcellulose, shellac, zein, natural or synthetic wax, hydrogenated vegetable oil, hydrogenated castor oil, polyvinylpyrrolidine, a vinyl acetate copolymer, a vinyl alcohol copolymer, polyethylene oxide, or a combination comprising at least one of the foregoing materials. The controlled-release oral composition can contain about 1.0 wt % to about 90 wt % of the release-retarding matrix material(s) based on the total weight of the oral composition, specifically about 5 wt % to about 80 wt %, and more specifically about 10 wt % to about 75 wt %.

Examples of suitable acrylic polymers that can be used as release-retarding matrix material(s) include, but are not limited to, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, or a combination comprising at least one of the foregoing materials. The acrylic polymer may comprise methacrylate copolymers described in NF XXIV as fully polymerized copolymers of acrylic and methacrylic acid esters with a low content of quaternary ammonium groups. Examples of copolymers of acrylic and methacrylic acid esters with a low content of quaternary ammonium groups useful in the present invention include EUDRAGIT RS, and EUDRAGIT RL commercially available from Rohm Pharma GmbH, Germany. Other examples of suitable copolymers of acrylic and methacrylic acid esters which can be used include EUDRAGIT NE30D, EUDRAGIT L, EUDRAGIT S, EUDRAGIT FS30D and the like, all of which are commercially available from Rohm Pharma GmbH, Germany.

Examples of alkylcelluloses that can be used in the present invention include, but are not limited to, methylcellulose, ethylcellulose, and the like. Those skilled in the art, upon reading this disclosure, will appreciate that other cellulosic polymers, including other alkyl cellulosic polymers, can be substituted for part or all of the ethylcellulose. Other release-retarding matrix material(s) which can be used in the present invention include modified celluloses such as a carboxymethylcellulose, hydroxypropylmethylcellulose, a crosslinked sodium carboxymethylcellulose, hydroxymethyl cellulose, hydroxyethyl cellulose, hydroxypropyl cellulose, or a combination comprising at least one of the foregoing materials.

Examples of other release-retarding matrix material(s) which can be used in the present invention include a natural or synthetic wax, a fatty alcohol (such as lauryl, myristyl, stearyl, cetyl or specifically cetostearyl alcohol), a fatty acid, including fatty acid esters, fatty acid glycerides (mono-, di-, and tri-glycerides), a hydrogenated fat, a hydrocarbon wax, a normal wax, stearic acid, stearyl alcohol, hydrophobic and hydrophilic materials having hydrocarbon backbones, or a combination comprising at least one of the foregoing materials. Examples of suitable waxes include, but are not limited to, beeswax, glycowax, castor wax, carnauba wax and wax-like substances, e.g., material normally solid at room temperature and having a melting point of from about 30° C. to about 100° C., or a combination comprising at least one of the foregoing waxes or wax-like substances.

Examples of other release-retarding matrix material(s) which can be used in the present invention include a digestible, long chain (e.g., C₈-C₅₀, specifically C₁₂-C₄₀) substituted or unsubstituted hydrocarbon, such as fatty acids, fatty alcohols, glyceryl esters of fatty acids, mineral and vegetable oils, waxes, or a combination comprising at least one of the foregoing materials. Hydrocarbons having a melting point of between about 25° C. and about 90° C. may be used. In one embodiment, long chain hydrocarbon materials, fatty (aliphatic) alcohols are used.

Still further examples of release-retarding matrix material(s) which can be used in the present invention include a polylactic acid, a polyglycolic acid, a co-polymer of lactic and glycolic acid, a carboxymethyl starch, a potassium methacrylate/divinylbenzene copolymer, a crosslinked polyvinylpyrrolidone, a polyvinylalcohol, a polyvinylalcohol copolymer, a polyethylene glycol, a non-crosslinked polyvinylpyrrolidone, a polyvinylacetate, a polyvinylacetate copolymer or a combination comprising at least one of the foregoing materials.

The release-retarding matrix material(s) can also optionally be combined with other additives such as, but not limited to, an erosion-promoting agent (e.g., starch and gums); or a semi-permeable polymer. In addition to the above ingredients, the controlled-release composition prepared from a controlled-release matrix may also contain suitable quantities of other materials, e.g., fillers diluents, disintegrants lubricants, binders, granulating aids, colorants, flavorants and glidants that are conventional in the pharmaceutical art.

Binders can be used to hold the ingredients in the composition together. Examples of binders include, but are not limited to, polyvinyl pyrrolidone, hydroxypropyl cellulose, hydroxypropyl methylcellulose, methylcellulose and hydroxyethyl cellulose, sugars, or a combination comprising at least one of the foregoing binders.

Disintegrants expand when wet and thus can be used to cause a tablet or other dosage form to break apart. Examples of disintegrants include, but are not limited to, water swellable substances such as low-substituted hydroxypropyl cellulose, e.g. L-HPC; cross-linked polyvinyl pyrrolidone (PVP-XL), e.g. Kollidon® CL and Polyplasdone® XL; cross-linked sodium carboxymethylcellulose (sodium croscarmellose), e.g. Ac-di-sol®, Primellose®; sodium starch glycolate, e.g. Primojel®; sodium carboxymethylcellulose; sodium carboxymethyl starch, e.g. Explotab®; ion-exchange resins, e.g. Dowex® or Amberlite®;

microcrystalline cellulose, e.g. Avicel®; starches and pregelatinized starch, e.g. Starch 1500®; formalin-casein, or a combination comprising at least one of the foregoing water swellable substances.

Lubricants can be used to aid in the processing of powder materials. Examples of lubricants include, but are not limited to, calcium stearate, glycerol behenate, magnesium stearate, mineral oil, polyethylene glycol, sodium stearyl fumarate, stearic acid, talc, vegetable oil, zinc stearate, or a combination comprising at least one of the foregoing lubricants.

A nonlimiting example of a glidant which can be optionally used is silicon dioxide.

Certain compositions described herein may also comprise a filler, such as a water insoluble filler, a water soluble filler, or a combination comprising at least one of the foregoing. Examples of water insoluble fillers include, but are not limited to, silicon dioxide, titanium dioxide, talc, alumina, starch, kaolin, polacrilin potassium, powdered cellulose, microcrystalline cellulose, sodium citrate, dicalcium phosphate or a combination comprising at least one of the foregoing fillers. Examples of water-soluble fillers include, but are not limited to, water soluble sugars and sugar alcohols, specifically lactose, glucose, fructose, sucrose, mannose, dextrose, galactose, the corresponding sugar alcohols and other sugar alcohols, such as mannitol, sorbitol, xylitol, or a combination comprising at least one of the foregoing fillers.

In addition to the SSRI and release-retarding material, the core of subunits used in the formulations of the present invention may also optionally further comprise an organic acid such as, but not limited to, adipic acid, ascorbic acid, citric acid, fumaric acid, malic acid, succinic acid and tartaric acid.

Optionally, certain compositions described herein may be coated with non-functional coatings. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

Generally, the controlled-release SSRI formulation comprises a SSRI and one or more release-retarding materials in the form of a matrix. The controlled-release formulation can be prepared in a monolithic form, a layered form comprising two or more layers or as a plurality of subunits encapsulated into a capsule.

By “controlled-release” is meant a dosage form in which the release of the active agent is controlled or modified over a period of time. Controlled can mean, for example, sustained-, delayed- or pulsed-release at a particular time. Alternatively, controlled can mean that the release of the active agent is extended for longer than it would be in an immediate-release dosage form, e.g., at least over several hours. In one embodiment of the present invention, the controlled-release composition exhibits not more than about 80% selective serotonin reuptake inhibitor (SSRI) released in 5 hours; and not less than about 80% selective serotonin reuptake inhibitor (SSRI) released in 18 hours. In one embodiment, the controlled-release composition is tested using a USP Type II apparatus, at 75 rpm, 900 mL of pH 6.8 phosphate buffer.

In one embodiment of the present invention, a tablet or capsule containing the subunits of the controlled-release formulation is administered.

In another embodiment of the present invention, the controlled-release formulation of the present invention is combined in a single dosage form with an optional immediate-release portion of an active agent in the form of immediate-release subunits (e.g., in a capsule or compressed into a tablet using compressible binders), immediate-release powders of the active agent, or immediate-release coatings of the active agent substantially surrounding the controlled-release subunits. By “immediate-release” it is meant a conventional or non-modified release in which greater than or equal to about 75% of the active agent is released within two hours of administration. The immediate-release portion of a combination dosage form may be provided as a loading dose.

In another embodiment of the present invention, two or more types of controlled-release subunits can be combined in one composition, where each subunit provides a different release profile (e.g., a combination of extended-release and modified-release subunits in a single formulation, optionally in combination with an immediate-release portion as described supra).

By “rate-controlling polymer coating” is meant a coating that extends the release of the active agent from the dosage form in a controlled manner over a period of time. Controlled can mean, for example, sustained-, delayed- or pulsed-release independent of the pH environment. Alternatively, controlled can mean that the release of the active agent is extended for longer than it would be in an immediate-release dosage form, e.g., at least over several hours. As will be understood by the skilled artisan upon reading this disclosure, rate-controlling polymer coating does not encompass enteric coating. While the enteric coating dissolves at a specified pH range presumably releasing the entire active agent from the dosage form immediately, rate-controlling polymer coatings control the release of the active agent independent of the pH environment.

In one embodiment of the present invention, the composition is coated with an enteric coating. Examples of enteric coatings which dissolve at a specified pH range include, but are not limited to, polymers such as methacrylic acid-ethyl acrylate copolymer (1:1), ethacrylic acid-methyl methacrylate copolymer (1:1), methacrylic acid-methyl methacrylate copolymer (1:2), polyvinyl acetate phthalate (PVAP), hydroxypropyl methylcellulose acetate succinate (HPMCAS) and cellulose acetate phthalate (CAP). Additionally, dyestuffs or pigments can be added to the enteric polymer coating for product identification or to characterize the quantity of active compound, i.e., dosage.

In some embodiments, prior to applying the enteric polymer coating, the composition is coated with a subcoating and then coated with the enteric coating to avoid drug interactions with the enteric coating. Examples of subcoatings include, but are not limited to polymers such as polyvinyl pyrrolidine, hydroxymethyl cellulose, hydroxypropylmethylcellulose, and hydroxypropylcellulose.

The controlled-release formulations of the present invention can be used to treat the symptoms of depression and in the treatment of obsessive compulsive disorder.

The controlled-release composition comprising selective serotonin reuptake inhibitor (SSRI) and a release-retarding matrix material(s) may be prepared by, for example, dry granulation or wet granulation followed by compression or compaction, melt extrusion and spheronization, and the like. Examples of such techniques include, but are not limited to, direct compression, using appropriate punches and dies which are fitted to a suitable rotary tableting press; injection or compression molding using suitable molds fitted to a compression unit, granulation followed by compression; and extrusion in the form of a paste, into a mold or to an extrudate to be cut into lengths.

Subunits for use in the present invention can also be prepared by compression into a compressed form (e.g., microtablets or minitablets) using conventional tableting equipment and standard techniques such as described in Remington's Pharmaceutical Sciences, (Aurther Osol., editor), 1553-1593 (1980).

The core of a SSRI or a pharmaceutically acceptable salt thereof of subunits used in the present invention can have a diameter of about 250 to about 7500 micrometers, specifically about 400 to about 5000 micrometers, and yet more specifically about 500 to about 4500 micrometers.

The selective serotonin reuptake inhibitor (SSRI) can be processed with a release-retarding matrix material(s) and formed into a plurality of subunits. In one embodiment, the formulation may comprise a plurality of subunits with the same controlled-release profile. In another embodiment, the formulation comprises a plurality of subunits, each exhibiting different characteristics, such as, but not limited to, pH dependent release, pH independent release, timed release in various media (e.g., acid, base, simulated intestinal fluid), release in vivo, size, and composition. The different subunits can then be combined to result in a composition formulated to meet a targeted release profile in vivo or in vitro. Immediate-release subunits can also be combined with controlled-release subunits into a single composition.

The subunits can be presented in a capsule as granules, pellets, microtablets or minitablets, blended with a compressible binder and compressed into tablets, or prepared into other suitable unit dosage forms.

In order to achieve the desired pharmacokinetic profile, the compositions may comprise subunits that release selective serotonin reuptake inhibitor (SSRI) at different rates. For example, a composition may comprise subunits that release selective serotonin reuptake inhibitor (SSRI) slowly, and subunits that release selective serotonin reuptake inhibitor (SSRI) more rapidly, in particular one type of subunit that releases the active ingredient immediately, e.g. subunits of SSRI as described that lack the release-retarding material(s).

In one embodiment, part of the total amount of selective serotonin reuptake inhibitor (SSRI) in the composition is present in an immediate-release portion. Any type of immediate-release compositions are contemplated, for example, as subunits lacking a release-retarding material(s) coating/matrix, immediate-release selective serotonin reuptake inhibitor (SSRI) powder or subunits which can be blended with controlled-release subunits, or as a topcoat coating the controlled-release subunits or coating the entire controlled-release composition.

The different subunits may be filled consecutively in the capsules, or they may be premixed and the resulting premix may be filled into the capsules (taking into account possible segregation).

Alternatively, the controlled-release subunits may further comprise a top-coat of a water-soluble non-rate controlling polymer and selective serotonin reuptake inhibitor (SSRI) which is released essentially immediately upon ingestion and thus ensures a rapid onset of action.

Alternatively, in embodiments of microtablets, minitablets or microcaplets, the controlled-release subunits may further comprise a second layer, as seen in a bi-layer tablet, which contains the selective serotonin reuptake inhibitor (SSRI) which is released essentially immediately upon ingestion and thus ensures a rapid onset of action.

The following nonlimiting examples are provided to further illustrate the present invention.

### EXAMPLES

### Example 1

### Controlled-Release Subunits: Compression

Controlled-release subunits in accordance with the present invention can be prepared by a granulation process followed by compression of the components listed in Table 1.

TABLE 1

| Component | % W/W |
|---|---|
| Fluvoxamine Maleate | 33.4 |
| Carnaubawax | 50 |
| Ethyl Cellulose | 5 |
| Eudragit RS PO | 10 |
| SD3A Alcohol | * |
| Silicon Dioxide (Syloid 244 FP) | 0.8 |
| Mg. Stearate | 0.8 |
| Total | 100 |

* not present in final formulation

The components, except Syloid and Magnesium stearate, are wet granulated with SD3A alcohol in a high shear granulator to form a wet granulation mixture. The wet granulation mixture is screened, dried, and milled. Syloid and Magnesium stearate are added to the granules and blended. The resulting blend is compressed into minitablets (mean diameter of about 2 to about 5 mm) or microtablets (mean length of about 2 to about 7 mm) each containing about 1 to about 10 mg of Fluvoxamine maleate per minitablet or microtablet.

### Example 2

### Controlled-Release Subunits: Compression

Controlled-release subunits in accordance with the present invention can also be prepared by a granulation process followed by compression of the components listed in Table 2.

TABLE 2

| Component | % W/W |
|---|---|
| Fluvoxamine Maleate | 33.4 |
| Carnaubawax | 45 |
| Ethyl Cellulose | 5 |
| Eudragit L100 | 5 |
| Eudragit RS PO | 10 |
| SD3A Alcohol | * |
| Silicon Dioxide (Syloid 244 FP) | 0.8 |
| Mg. Stearate | 0.8 |
| Total | 100 |

* not present in final formulation

The components, except Syloid and Magnesium stearate, are wet granulated with SD3A alcohol in a high shear granulator to form a wet granulation mixture. The wet granulation mixture is screened, dried, and milled. Syloid and Magnesium stearate are added to the granules and blended. The resulting blend is compressed into minitablets (mean diameter of about 2 to about 5 mm) or microtablets (mean length of about 2 to about 7 mm) each containing about 1 to about 10 mg of Fluvoxamine maleate per minitablet or microtablet.

### Example 3

### Controlled-Release Subunits: Compression

Controlled-release subunits in accordance with the present invention can be prepared by a granulation process followed by compression of the components listed in Table 3.

TABLE 3

| Component | % W/W |
|---|---|
| Fluvoxamine Maleate | 33.4 |
| Carnaubawax | 50.8 |
| Ethyl Cellulose | 5 |
| Eudragit RS PO | 5 |
| Eudragit RL PO | 5 |
| SD3A Alcohol | * |
| Mg. Stearate | 0.8 |
| Total | 100 |

* not present in final formulation

The components, except Syloid and Magnesium stearate, are wet granulated with SD3A alcohol in a high shear granulator to form a wet granulation mixture. The wet granulation mixture is screened, dried, and milled. Syloid and Magnesium stearate are added to the granules and blended. The resulting blend is compressed into minitablets (mean diameter of about 2 to about 5 mm) or microtablets (mean length of about 2 to about 7 mm) each containing about 1 to about 10 mg of Fluvoxamine maleate per minitablet or microtablet.

### Example 4

### Controlled-Release Subunits: Extrusion/Spheronization

The formulations of Examples 1, 2 and 3 can also be prepared as controlled-release pellets in accordance with the present invention. Spherical pellets can be prepared using extrusion/spheronization techniques well known in the pharmaceutical art. If necessary, additional small amounts of the release-retarding matrix material(s) or the binder can be added to aid in the spheronization process.

### Example 5

### Dissolution Profiles for Controlled-Release Subunits

Controlled-release subunits are prepared as described in Example 1 to 4. A number of subunits equaling 100 mg of Fluvoxamine maleate are taken from each sample and tested in 900 ml of pH 6.8 phosphate buffer dissolution medium, using paddles rotating at 75 rpm, at temperature of 37° C.±2° C.

1. A composition of controlled-release selective serotonin reuptake inhibitor (SSRI) containing one or more subunits, wherein each subunit comprises a selective serotonin reuptake inhibitor (SSRI) and a release-retarding material.

2. The composition of claim 1 wherein the subunit is not coated with a rate-controlling polymer.

3. The composition of claim 1 coated with an enteric coating and an optional subcoating between the composition and the enteric coating.

4. The composition of claim 1, wherein the SSRI is selective serotonin reuptake inhibitor (SSRI) free base, selective serotonin reuptake inhibitor (SSRI) benzoate, selective serotonin reuptake inhibitor (SSRI) citrate, selective serotonin reuptake inhibitor (SSRI) glutarate, selective serotonin reuptake inhibitor (SSRI) hydrobromide, selective serotonin reuptake inhibitor (SSRI) hydrochloride, selective serotonin reuptake inhibitor (SSRI) hydrogen phosphate, selective serotonin reuptake inhibitor (SSRI) dihydrogen phosphate, selective serotonin reuptake inhibitor (SSRI) lactate, selective serotonin reuptake inhibitor (SSRI) mandelate, selective serotonin reuptake inhibitor (SSRI) maleate, selective serotonin reuptake inhibitor (SSRI) mesylate, selective serotonin reuptake inhibitor (SSRI) oxalate, selective serotonin reuptake inhibitor (SSRI) sulfate, or a hydrate, solvate, polymorph, or non-crystalline form of the foregoing.

5. The composition of claim 1, wherein the selective serotonin reuptake inhibitor (SSRI) is fluvoxamine maleate.

6. The composition of claim 1 wherein the release-retarding material is a release-retarding matrix material and not a rate controlling polymer coating.

7. The composition of claim 6 wherein the release-retarding matrix material is an acrylic polymer, a cellulose, a wax, a fatty acid, shellac, zein, hydrogenated vegetable oil, hydrogenated castor oil, polyvinylpyrrolidine, a vinyl acetate copolymer, a vinyl alcohol copolymer, polyethylene oxide, or a combination comprising at least one of the foregoing materials.

8. The composition of claim 6 wherein the release-retarding matrix material is an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, or a combination comprising at least one of the foregoing materials.

9. The composition of claim 6, wherein the release-retarding matrix material is a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, or a combination comprising at least one of the foregoing materials.

10. The composition of claim 6, wherein the release-retarding matrix material is a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax or a combination comprising at least one of the foregoing materials.

11. The composition of claim 6, wherein the release-retarding matrix material is polylactic acid, polyglycolic acid, a co-polymer of lactic and glycolic acid, carboxymethyl starch, potassium methacrylate/divinylbenzene copolymer, crosslinked polyvinylpyrrolidone, polyvinylalcohols, polyvinylalcohol copolymers, polyethylene glycols, non-crosslinked polyvinylpyrrolidone, polyvinylacetates, polyvinylacetate copolymers or a combination comprising at least one of the foregoing materials.

12. The composition of claim 1, wherein the composition comprises a plurality of controlled-release subunits comprising the SSRI and the release-retarding material.

13. The composition of claim 12 wherein the controlled-release subunits are extended-release or delayed-release.

14. The composition of claim 12 wherein the controlled-release subunits have a mean diameter or mean length of greater than about 250 micrometers.

15. The composition of claim 12 wherein the controlled-release subunits have a mean diameter or mean length of greater than about 1500 micrometers.

16. The composition of claim 12 wherein the controlled-release subunits have a mean diameter of about 250 to about 7500 micrometers or mean length of about 250 to about 1 centimeter.

17. The composition of claim 12 wherein the controlled-release subunits have a mean diameter or mean length of about 400 to about 7500 micrometers.

18. The composition of claim 12 wherein the controlled-release subunits have a mean diameter or mean length of about 500 to about 5000 micrometers.

19. The composition of claim 12, wherein the controlled-release subunits are prepared by granulation, compression, compaction, extrusion, spheronization or extrusion and spheronization.

20. The composition of claim 12, wherein the controlled-release subunits are prepared by compression.

21. The composition of claim 20, wherein the composition is a capsule comprising controlled-release subunits.

22. The composition of claim 20, wherein the composition is a tablet comprising controlled-release subunits.

23. The composition of claim 1, wherein the composition further comprises an immediate-release portion.

24. The composition of claim 23 wherein the immediate-release portion comprises a SSRI and a pharmaceutical excipient.

25. The composition of claim 23 wherein the immediate-release portion comprises a plurality of immediate-release subunits comprising a SSRI and a pharmaceutical excipient.

26. The composition of claim 23, wherein the composition is a capsule comprising immediate-release subunits and controlled-release subunits.

27. The composition of claim 23, wherein the composition is a tablet comprising immediate-release subunits and controlled-release subunits.

28. A method of treating a symptom of depression or obsessive compulsive disorder by administering a controlled-release SSRI composition according to claim 1.
